(19) United States
(12) Patent Application Publication
(10) Pub. No.: US 2026/0151180 A1
(43) Pub. Date: Jun. 4, 2026

(54) MEDICAL DEVICE

(71) Applicant: TERUMO KABUSHIKI KAISHA, TOKYO (JP)

(72) Inventor: YUSAKU YAMAKI, KANAGAWA (JP)

(21) Appl. No.: 19/454,629

(22) Filed: Jan. 21, 2026

Related U.S. Application Data
(63) Continuation of application No. PCT/JP2024/026242, filed on Jul. 23, 2024.

(30) Foreign Application Priority Data
Aug. 25, 2023 (JP) 2023137190

(51) Int. Cl. A61B 18/14; A61B 17/00; A61B 17/29; A61B 17/295; A61B 18/00

(52) U.S. Cl. CPC A61B 18/1445; A61B 17/295; A61B 2017/00398; A61B 2017/00438; A61B 2017/00778; A61B 2017/2941; A61B 2017/2946; A61B 2018/00428; A61B 2018/00595; A61B 2018/0094; A61B 2018/1455; A61B 2018/1467

(57) ABSTRACT

This medical device (10) includes an operation unit (100), a first jaw (250a) and a second jaw (250b) that are provided at the distal end of a shaft member (230), and a power transmission member (300). The operation unit (100) has a first operation lever (102a) and a second operation lever (102b). When a user rotates the first operation lever (102a) and the second operation lever (102b) with respect to a casing (20), the power transmission member moves along the axial direction of the shaft member. With this movement, the first jaw and the second jaw are opened and closed.

### CROSS REFERENCE TO RELATED APPLICATIONS

This application is a continuation of PCT Application PCT/JP 2024/026242, filed on Jul. 23, 2024, based on and claiming priority to Japanese Application No. JP 2023137190, filed on Aug. 25, 2023, both of which are incorporated herein by reference in their entirety.

### BACKGROUND OF THE INVENTION

The present invention relates to a medical device for harvesting biological tissue.

In coronary artery bypass surgery, a blood vessel bypassing a lesion site is connected to an artery. The blood vessel is harvested from, for example, a lower limb of a patient by an endoscopic vessel harvesting system.

The endoscopic vessel harvesting system includes a medical device including a vessel harvesting tool. As the vessel harvesting tool, a configuration having openable and closable first and second jaws is conceivable as described in Japanese Patent No. 6840749 and Japanese Patent No. 6855457. In this configuration, the blood vessel is pinched between the first jaw and the second jaw by bringing the first jaw and the second jaw into a closed state from an open state in the body of the patient. Next, the vessel harvesting tool is moved to the outside of the patient's body, whereby the blood vessel pinched between the first jaw and the second jaw is extracted outside the body. In this case, the biological tissue harvested by the medical device is a blood vessel.

In the medical device described in Japanese Patent No. 6840749, a casing accommodating an ultrasound transducer assembly is provided with a first jaw and a first ring into which a user inserts his/her finger. On the other hand, a second jaw and a second ring are provided on a shank rotatably connected to the casing. Therefore, when the user opens and closes the first jaw and the second jaw by putting his/her fingers through the first ring and the second ring, the shank and the casing may shake in accordance with the movement of the fingers of the user.

Further, the rings into which the user inserts his/her fingers are close to the first jaw and the second jaw. Therefore, in this medical device, the first jaw and the second jaw cannot be inserted deep into the patient's body.

In the medical device described in Japanese U.S. Patent No. 6855457, a first jaw and a second jaw can be inserted deep into the body of a patient. In this medical device, the first jaw and the second jaw are opened and closed by simultaneously pushing two levers toward a cylindrical casing. Therefore, the user needs to grip the casing and simultaneously hold down the two levers with two fingers, but in this case, it is not easy to smoothly open and close the first jaw and the second jaw.

### SUMMARY OF THE INVENTION

It is therefore an object of the present invention to solve the above-described problems.

An aspect of the present invention provides a medical device including: an operation unit operated by a user; a shaft member extending in a distal direction from the operation unit; and a first jaw and a second jaw provided to be openable and closable at a distal end of the shaft member. The operation unit includes: a casing; a first operation lever rotatable with respect to the casing and having a first finger hook portion that is annular and that is formed with a first finger hole into which a finger of the user is inserted; and a second operation lever rotatable with respect to the casing and having a second finger hook portion that is annular and that is formed with a second finger hole into which a finger of the user different from the finger to be inserted into the first finger hole is inserted. The first operation lever and the second operation lever face each other across the casing.

The medical device includes a power transmission member that is movable along an axial direction of the shaft member and that rotates at least one of the first jaw and the second jaw in such a manner that the first jaw and the second jaw are opened and closed with the movement in the axial direction with respect to the shaft member. The power transmission member moves in the axial direction with respect to the shaft member in conjunction with rotation operation of the first operation lever and the second operation lever with respect to the casing.

The user inserts his/her fingers into the first operation lever and the second operation lever, respectively, while holding (gripping) the casing in the palm, and with this state, moves the fingers to apply rotational force to the first operation lever and the second operation lever, by which the first jaw and the second jaw can be opened and closed. The user can easily put strength in his/her hand and can easily move the fingers, whereby the operability of the medical device is improved.

In the medical device according to the first aspect of the present invention, as the first operation lever rotates in such a manner that the first finger hook portion approaches the casing, and the second operation lever rotates in such a manner that the second finger hook portion approaches the casing, the power transmission member may move toward the distal end along the axial direction of the shaft member, and the first jaw and the second jaw may be in a closed state. On the other hand, as the first operation lever rotates in such a manner that the first finger hook portion is separated from the casing, and the second operation lever rotates in such a manner that the second finger hook portion is separated from the casing, the power transmission member may move toward a proximal end along the axial direction of the shaft member, and the first jaw and the second jaw may be in an open state.

In this case, the open state and the closed state of the first operation lever and the second operation lever match the open state and the closed state of the first jaw and the second jaw. Therefore, the user can easily recognize that the first jaw and the second jaw have been changed from the open state to the closed state by, for example, operating the first operation lever and the second operation lever to shift them from the open state to the closed state.

In the medical device according to above noted aspects of the present invention, rotation centers of the first operation lever and the second operation lever may be provided at a proximal end portion of the casing. The first operation lever and the second operation lever may rotate in such a manner that the first finger hook portion and the second finger hook portion are separated from or approach the casing on a distal side with respect to the rotation centers.

In this case, the user can easily rotate the first operation lever and the second operation lever.

In the medical device according to above noted aspects of the present invention, the operation unit may include: a first toggle mechanism connected to the casing, the first operation lever, and the power transmission member; and a second toggle mechanism connected to the casing, the second operation lever, and the power transmission member. The first toggle mechanism may include a first link and a second link rotatably connected to each other. The first link may include a first end rotatably connected to the casing and a second end rotatably connected to the second link, and the second link may include a third end rotatably connected to the second end and a fourth end rotatably connected to the power transmission member. The first operation lever may be connected to a connection point between the second end and the third end.

The second toggle mechanism may include a third link and a fourth link rotatably connected to each other. The third link may include a fifth end rotatably connected to the casing and a sixth end rotatably connected to the fourth link, and the fourth link may include a seventh end rotatably connected to the sixth end and an eighth end rotatably connected to the power transmission member. The second operation lever may be connected to a connection point between the sixth end and the seventh end.

The power transmission member constitutes the first toggle mechanism with the first link and the second link, and constitutes the second toggle mechanism with the third link and the fourth link. Therefore, the user can easily move the power transmission member by rotating the first operation lever and the second operation lever with a small force.

The medical device according to the above noted aspects of the present invention may further include a cutter that excises biological tissue. In this aspect, the operation unit may include a cutter moving operation member for moving the cutter along the axial direction of the shaft member in the casing. The cutter moving operation member may be located distal to the first finger hook portion or the second finger hook portion. The casing may have a first side surface and a second side surface located opposite to each other with respect to an axis of the casing, the first operation lever may be disposed on the first side surface, the second operation lever may be disposed on the second side surface, and the cutter moving operation member may be disposed on the first side surface or the second side surface.

Since it is possible to excise the biological tissue by the cutter, it is easy to harvest the biological tissue. In addition, the user can easily touch the cutter moving operation member with a finger not inserted into the first finger hole and not inserted into the second finger hole while gripping the casing with the palm. Therefore, the user can easily move the cutter.

In the medical device according to above noted aspects of the present invention, the cutter moving operation member may be a lever. In this aspect, one end of the lever may be connected to the casing via a support member on a distal side with respect to the first finger hook portion or the second finger hook portion, the lever may be rotatable with the support member as a rotation center in such a manner that a free end which is another end of the lever approaches or is separated from the casing, and the cutter may move toward the distal end along the axial direction of the shaft member by rotating the lever in such a manner that the free end of the lever approaches the casing.

As described above, the user can easily touch the lever with a finger that is not inserted into the first finger hole and is not inserted into the second finger hole. Moreover, one end of the lever facing the distal end of the casing is connected to the casing, and the other end facing the proximal end of the casing is located on the distal side of the casing with respect to the first finger hook portion or the second finger hook portion. Therefore, the lever can be easily rotated by pushing the other end of the lever toward the casing with the finger. Therefore, the user can easily move the cutter.

In the medical device according to above noted aspects of the present invention, the first jaw and the second jaw may include a first electrode and a second electrode, respectively, for cauterizing biological tissue. In this aspect, the operation unit may include a changeover switch that switches energization or deenergization between the first electrode and the second electrode in the casing, the changeover switch may be located distal to the first finger hook portion or the second finger hook portion, the casing may have a first side surface and a second side surface located opposite to each other with respect to an axis of the casing, the first operation lever may be disposed on the first side surface, the second operation lever may be disposed on the second side surface, and the changeover switch may be disposed on the first side surface or the second side surface.

By energizing the first electrode and the second electrode, it is possible to cauterize the biological tissue pinched between the first jaw and the second jaw. Therefore, when the biological tissue is, for example, a blood vessel, hemostasis can be promptly performed when the blood vessel is harvested. In addition, the user can easily touch the changeover switch with a finger not inserted into the first finger hole and not inserted into the second finger hole while gripping the casing with the palm. Therefore, the user can easily switch the energization or deenergization between the first electrode and the second electrode.

In the medical device according to above noted aspects of the present invention, the changeover switch may be movable toward the distal end or the proximal end along the axial direction of the shaft member.

With this configuration, the user can easily switch energization or deenergization between the first electrode and the second electrode by moving the changeover switch toward the distal end or the proximal end along the axial direction of the casing with the finger not inserted into the first finger hole and not inserted into the second finger hole.

The medical device according to the above noted aspects of the present invention may include a cutter that excises biological tissue, and the first jaw and the second jaw may include a first electrode and a second electrode, respectively, for cauterizing the biological tissue. In this aspect, the operation unit may include, in the casing, a changeover switch that switches energization or deenergization between the first electrode and the second electrode, and a cutter moving operation member for moving the cutter along the axial direction of the casing, the changeover switch may be located distal to the first finger hook portion or the second finger hook portion, and the cutter moving operation member may be located distal to the first finger hook portion or the second finger hook portion. The casing may have a first side surface and a second side surface located opposite to each other with respect to an axis of the casing, the first operation lever may be disposed on the first side surface, the second operation lever may be disposed on the second side surface, the changeover switch may be disposed on one of the first side surface and the second side surface, and the cutter moving operation member may be disposed on the other of the first side surface and the second side surface.

With this configuration, it is possible to excise the biological tissue by the cutter, whereby it is easy to harvest the biological tissue. In addition, it is possible to cauterize the biological tissue pinched between the first jaw and the second jaw, and thus, in a case where the biological tissue is, for example, a blood vessel, hemostasis can be quickly performed when the blood vessel is harvested.

Moreover, the user can individually touch the cutter moving operation member or the changeover switch with the finger not inserted into the first finger hole and not inserted into the second finger hole while holding the casing with the palm. Therefore, the user can easily move the cutter, and can easily switch the energization or the deenergization between the first electrode and the second electrode.

In the medical device according to above noted aspects of the present invention, the casing may have, at a proximal end, an insertion hole through which a first power line electrically connected to the first electrode and a second power line electrically connected to the second electrode pass.

The proximal end of the casing is a portion that is not touched by a user's finger or palm. Therefore, by passing the first power line and the second power line through the insertion hole formed at the above position, the first power line and the second power line are prevented from interfering with the user's operation when the user operates the medical device.

In the medical device according to above noted aspects of the present invention, the operation unit may include a stopper that stops movement of the power transmission member toward a proximal end of the casing, the stopper being located proximal to a proximal end of the power transmission member inside the casing.

When the power transmission member comes into contact with the stopper, the opening operation (or the closing operation) of the first jaw and the second jaw is stopped. Therefore, for example, the first jaw and the second jaw are avoided from being excessively opened, whereby it is easy to insert the first jaw and the second jaw into the body of a living body.

The medical device according to the above noted aspects of the present invention, the operation unit may include a coil spring that biases the power transmission member in the distal direction inside the casing.

For example, when the user removes his/her fingers from the first finger hole and the second finger hole after the first operation lever and the second operation lever are rotated from the closed state to the open state by the user's operation, the coil spring pushes the power transmission member based on elasticity. As a result, the first operation lever and the second operation lever are changed from the open state to the closed state. Therefore, the user does not need to perform an operation for returning the first operation lever and the second operation lever from the open state to the closed state.

In the medical device according to above noted aspects of the present invention, the first jaw and the second jaw may include a first electrode and a second electrode, respectively, for cauterizing biological tissue, the stopper may have a through hole through which a first power line electrically connected to the first electrode and a second power line electrically connected to the second electrode pass, and the casing may have an insertion hole through which the first power line and the second power line pass, the insertion hole being located proximal to a proximal end of the stopper.

With this configuration, the first power line and the second power line can pass through the insertion hole formed on the proximal side with respect to the stopper via the through hole of the stopper. Therefore, even though the stopper is provided inside the casing, the first power line and the second power line can be exposed from the inside to the outside of the casing.

In the medical device according to above noted aspects of the present invention, the shaft member may be a hollow body, and a part of the power transmission member may be inserted into the shaft member so as to be relatively movable with respect to the shaft member.

Since a part of the power transmission member is inserted into the shaft member, the medical device can be downsized.

According to the present invention, the user can open and close the first jaw and the second jaw by moving his/her fingers. This improves the operability of the medical device.

### BRIEF DESCRIPTION OF THE DRAWINGS

FIG. 1 is a schematic overall side view of a medical device according to an embodiment of the present invention.

FIG. 2 is an exploded perspective view of the medical device.

FIG. 3 is a schematic perspective view of an operation unit without illustrating a second half body constituting the casing.

FIG. 4 is an enlarged side view of a main part of the operation unit in which the periphery of a cutter moving operation member is enlarged.

FIG. 5 is an enlarged side view of a main part of the operation unit in which the periphery of a changeover switch is enlarged.

FIG. 6 is an enlarged perspective view of a main part of the distal end of the medical device.

FIG. 7 is an enlarged perspective view of the main part without illustrating the shaft member illustrated in FIG. 6.

FIG. 8 is a schematic side view of a first jaw and a second jaw in an open state.

FIG. 9 is a side view of a main part of the operation unit when a first operation lever and a second operation lever are in a closed state.

FIG. 10 is an enlarged side view of a main part of the operation unit when the changeover switch illustrated in FIG. 5 is operated.

FIG. 11 is an enlarged side view of a main part of the operation unit when the cutter moving operation member illustrated in FIG. 4 is operated.

FIG. 12 is a schematic side view illustrating a state in which the first jaw and the second jaw are in a closed state and a cutter is advanced.

### DETAILED DESCRIPTION OF PREFERRED EMBODIMENTS

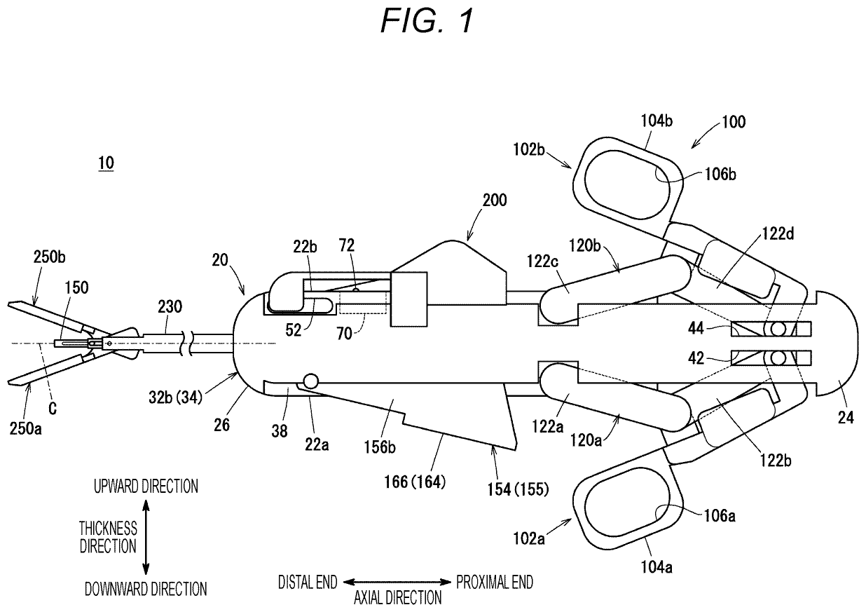

In the following description, the term “distal end” refers to, for each component, a direction approaching biological tissue when the biological tissue is harvested. The term “proximal end” refers to, for each component, an opposite direction to the distal end. For example, in a medical device 10 illustrated in FIG. 1, a first jaw 250a and a second jaw 250b are positioned at the distal end relative to a shaft member 230, and an operation unit 100 is positioned at the proximal end relative to the shaft member 230.

The “axial direction” is a direction connecting the distal end and the proximal end, and is a direction along a central axis C of the shaft member 230. The “distal end” (or “distal direction”) refers to an orientation (or direction) toward biological tissue in the axial direction when the biological tissue is harvested. The “proximal end” (or “proximal direction”) refers to an orientation (or direction) opposite to the distal end in the axial direction, and is an orientation (or direction) away from the biological tissue when the biological tissue is harvested. As understood from FIG. 1, the direction (axial direction) in which the central axis C of the shaft member 230 extends is parallel to the direction in which the axis of the casing 20 extends.

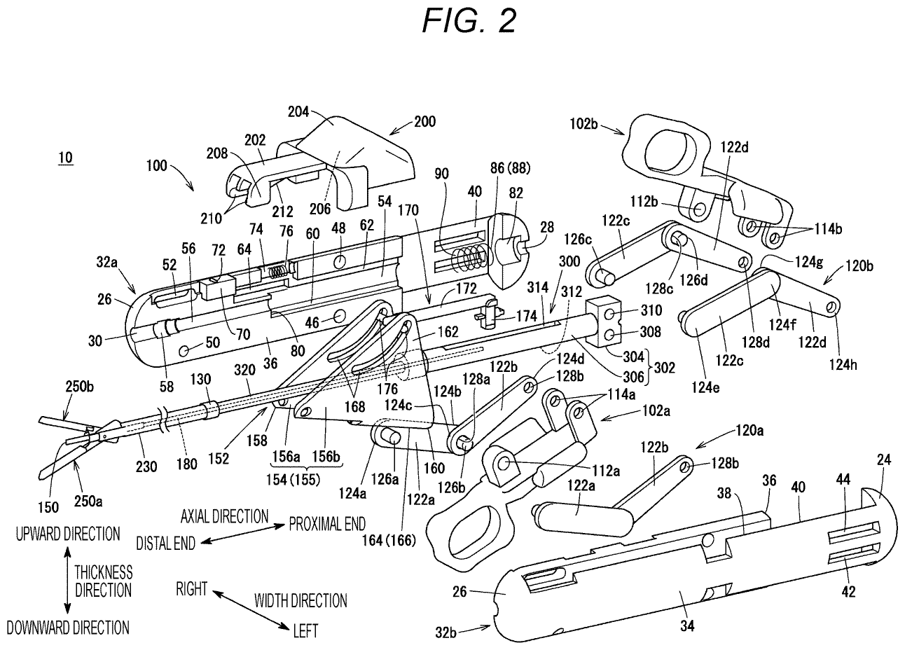

FIG. 1 is a schematic overall side view of the medical device 10 according to the present embodiment. FIG. 2 is an exploded perspective view of the medical device 10. The medical device 10 includes the operation unit 100, the shaft member 230, a power transmission member 300, the first jaw 250a, and the second jaw 250b.

The operation unit 100 includes the casing 20. As illustrated in FIG. 1, the casing 20 has a substantially oval shape when viewed from the width direction orthogonal to the axial direction and the thickness direction. The casing 20 has a first side surface 22a and a second side surface 22b located opposite to each other with respect to the axis of the casing 20. Here, the width direction is a horizontal direction orthogonal to the axial direction. When the user views the distal end from the proximal end of the operation unit 100, the right hand side of the user is defined as the right, and the left hand side of the user is defined as the left. The thickness direction is a direction connecting the first side surface 22a and the second side surface 22b. The “upward direction” is one direction in the thickness direction, and is a direction from the first side surface 22a toward the second side surface 22b. The “downward direction” which is one direction in the thickness direction is a direction opposite to the upward direction and is a direction from the second side surface 22b toward the first side surface 22a.

A proximal end of the casing 20 has a proximal-side dome portion 24, and a distal end of the casing 20 has a distal-side dome portion 26. As illustrated in FIG. 2, the proximal-side dome portion 24 has an insertion hole 28, and the distal-side dome portion 26 has a first fixing hole 30. The insertion hole 28 and the first fixing hole 30 extend along the axial direction.

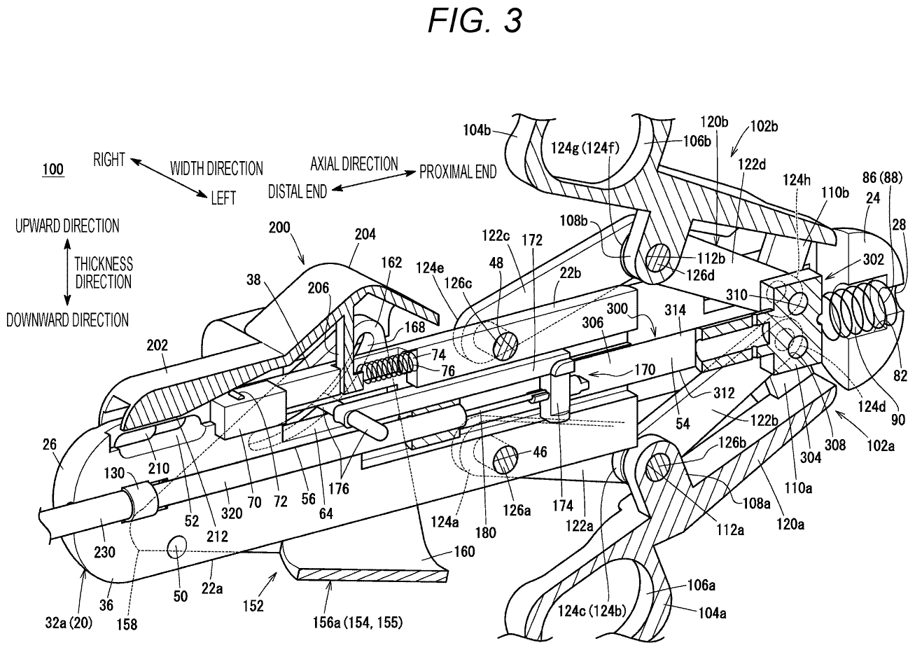

The casing 20 has a first half body 32a and a second half body 32b. The first half body 32a and the second half body 32b have shapes obtained by dividing the casing 20 into two along the thickness direction at the center in the width direction. Therefore, when the second half body 32b is removed from the first half body 32a, the proximal-side dome portion 24, the insertion hole 28, the distal-side dome portion 26, and the first fixing hole 30 are divided into two as illustrated in FIG. 3. Note that, in the following description, in order to simplify the description and facilitate understanding, the names of the components in the casing 20 are used for the names of the components of the first half body 32a and the second half body 32b. That is, each of the first half body 32a and the second half body 32b has the proximal-side dome portion 24, the insertion hole 28, the distal-side dome portion 26, and the first fixing hole 30.

FIG. 3 is a schematic perspective view of the operation unit 100 without illustrating the second half body 32b. As illustrated in FIGS. 2 and 3, the first half body 32a and the second half body 32b each have an outer plate 34 and an inner plate 36 in addition to the proximal-side dome portion 24 and the distal-side dome portion 26. Specifically, the proximal-side dome portion 24 and the distal-side dome portion 26 are connected to each other via the outer plates 34. The outer plate 34 extends along the axial direction. The proximal-side dome portion 24 and the distal-side dome portion 26 protrude inward of the casing 20 along the width direction from the proximal ends and the distal ends of the outer plates 34 in the axial direction, respectively. Therefore, the proximal-side dome portion 24 and the distal-side dome portion 26 are bent by approximately 90° with respect to the outer plates 34.

The inner plate 36 is connected to the end of the distal-side dome portion 26 facing the inside of the casing 20 in the width direction. The inner plate 36 extends from the distal end toward the proximal end so as to be aligned parallel to the outer plate 34. The outer plate 34 and the inner plate 36 are slightly separated from each other in the width direction. Due to the separation, a gap 38 is formed between the outer plate 34 and the inner plate 36. Here, the length along the axial direction of the inner plate 36 is smaller than the length along the axial direction of the outer plate 34. Therefore, in the first half body 32a, an internal space 40 surrounded by the end surface on the proximal side of the inner plate 36, the inner surface on the proximal side of the outer plate 34, and the inner surface of the proximal-side dome portion 24 is formed. The internal space 40 is in communication with the gap 38.

A first slit 42 and a second slit 44 are formed at the proximal end of the outer plate 34. The first slit 42 and the second slit 44 extend toward the distal end along the axial direction. The first slit 42 and the second slit 44 are in communication only with the internal space 40 and are not in communication with the gap 38.

The inner plate 36 has a first connection hole 46 and a second connection hole 48 at positions close to the proximal end, and has a support hole 50 and an engagement hole 52 at positions close to the distal-side dome portion 26. The first connection hole 46 and the second connection hole 48 are formed at line-symmetric positions with the center of the casing 20 in the thickness direction as a symmetry center. The support hole 50 and the engagement hole 52 are also line-symmetric with the center of the casing 20 in the thickness direction as a symmetry center. It is to be noted that the support hole 50 is a round hole formed at a position close to the first side surface 22a, whereas the engagement hole 52 is an elongated hole formed at a position close to the second side surface 22b. The first connection hole 46, the second connection hole 48, the support hole 50, and the engagement hole 52 penetrate along the width direction of the inner plate 36. In the outer plate 34, portions corresponding to the first connection hole 46, the second connection hole 48, the support hole 50, and the engagement hole 52 are cut out (see FIG. 2).

As described above, the inner surface of the distal-side dome portion 26 has the first fixing hole 30 extending along the axial direction. The inner plate 36 has, in the inner surface, a first guide hole 54, a second guide hole 56, and a second fixing hole 58 extending along the axial direction. The first guide hole 54, the second guide hole 56, the second fixing hole 58, and the first fixing hole 30 are arranged in this order from the proximal end toward the distal end in the axial direction. The first guide hole 54 and the first fixing hole 30 are connected via the second guide hole 56 and the second fixing hole 58. When the first half body 32a is viewed from the axial direction, the first guide hole 54, the second guide hole 56, the second fixing hole 58, and the first fixing hole 30 have a substantially semicircular shape opened inward in the width direction. The inner diameter of the first guide hole 54 is larger than the inner diameter of the second guide hole 56. Based on this inner diameter difference, a step portion 80 is formed at the boundary between the first guide hole 54 and the second guide hole 56. The inner diameter of the first fixing hole 30 is smaller than the inner diameter of the second fixing hole 58.

On the inner surface of the inner plate 36, the lower side and the upper side of the first guide hole 54 are notched stepwise. As a result, a first slide hole 60 is formed below the first guide hole 54, and a second slide hole 62 is formed above the first guide hole 54. Further, a third slide hole 64 is connected to the distal end of the second slide hole 62. The first slide hole 60, the second slide hole 62, and the third slide hole 64 extend along the axial direction. The depth of the third slide hole 64 along the width direction is larger than the depths of the first slide hole 60 and the second slide hole 62 along the width direction.

In the inner plate 36, a switch box 70 is positioned and fixed on the proximal side relative to the engagement hole 52. The switch box 70 has a button 72 on the top surface. In the inner plate 36, a first accommodation hole 74 is formed further on the proximal side relative to the switch box 70. A first return spring 76 is accommodated in the first accommodation hole 74.

The second half body 32b has a shape that is line-symmetric with the shape of the first half body 32a with the axis of the casing 20 as the symmetry center. For this reason, regarding the components of the second half body 32b, the same components as those of the first half body 32a are denoted by the same names and the same reference numerals, and the detailed description thereof will be omitted.

The power transmission member 300 includes a shaft-side slider 302 and a driven shaft 320. The driven shaft 320 will be described later.

The shaft-side slider 302 includes a block 304 having a substantially rectangular parallelepiped shape and a sleeve 306 having a substantially cylindrical shape. The block 304 faces the proximal end, and the sleeve 306 faces the distal end. The entire block 304 and the entire sleeve 306 are accommodated in the casing 20. The block 304 is disposed in the internal space 40. The block 304 has a third connection hole 308 and a fourth connection hole 310 extending along the width direction. The third connection hole 308 overlaps the first slit 42 in the width direction. The fourth connection hole 310 overlaps the second slit 44 in the width direction. The sleeve 306 extending along the axial direction is slidably inserted into the first guide hole 54. A first opening 312 facing the first side surface 22a of the casing 20 and a second opening 314 facing the second side surface 22b of the casing 20 are formed on the side surface of the sleeve 306. The first opening 312 and the second opening 314 extend along the axial direction.

The operation unit 100 includes a first operation lever 102a and a second operation lever 102b. The first operation lever 102a has an annular first finger hook portion 104a at the distal end. A first finger hole 106a is formed in the first finger hook portion 104a. The first operation lever 102a further includes a first tab 108a provided on the distal side close to the first finger hook portion 104a, and a pair of second tabs 110a provided on the proximal end away from the first finger hook portion 104a. The first tab 108a and the pair of second tabs 110a face the first side surface 22a of the casing 20. A fifth connection hole 112a is formed in the first tab 108a. A sixth connection hole 114a is formed in each of the pair of second tabs 110a.

The second operation lever 102b has an annular second finger hook portion 104b at the distal end. A second finger hole 106b is formed in the second finger hook portion 104b. The first operation lever 102a and the second operation lever 102b face each other across the casing 20. Specifically, the first operation lever 102a and the second operation lever 102b are rotationally symmetric with the central axis C of the shaft member 230 as a symmetry center.

The second operation lever 102b further includes a third tab 108b provided on the distal side close to the second finger hook portion 104b, and a pair of fourth tabs 110b provided on the proximal end away from the second finger hook portion 104b. The third tab 108b and the pair of fourth tabs 110b face the casing 20. A seventh connection hole 112b is formed in the third tab 108b. An eighth connection hole 114b is formed in each of the pair of fourth tabs 110b.

The operation unit 100 includes a first toggle mechanism 120a and a second toggle mechanism 120b. The first toggle mechanism 120a is coupled to the casing 20, the first operation lever 102a, and the shaft-side slider 302 constituting the power transmission member 300. Specifically, the first toggle mechanism 120a has a pair of left and right first links 122a and a pair of left and right second links 122b. The first link 122a on the left side and the second link 122b on the left side are rotatably connected to each other. The first link 122a on the right side and the second link 122b on the right side are rotatably connected to each other. Each of the pair of first links 122a has a first end 124a and a second end 124b, the first end 124a is provided with a pin-shaped first engagement protrusion 126a, and the second end 124b is provided with a pin-shaped second engagement protrusion 126b. Each of the pair of second links 122b has a third end 124c and a fourth end 124d, and a first coupling hole 128a is formed in the third end 124c. The first coupling hole 128a overlaps the fifth connection hole 112a in the width direction. A second coupling hole 128b is formed in the fourth end 124d. The fourth end 124d of the second link 122b is interposed between the second tab 110a of the first operation lever 102a and the block 304 of the shaft-side slider 302 in the width direction. Therefore, the sixth connection holes 114a at the second tabs 110a, the second coupling holes 128b at the fourth ends 124d, and the third connection hole 308 at the block 304 overlap each other in the width direction.

The first engagement protrusion 126a at the first end 124a of the first link 122a is rotatably engaged with the first connection hole 46 formed in the casing 20. The second engagement protrusion 126b at the second end 124b of the first link 122a is rotatably engaged with the first coupling hole 128a at the third end 124c of the second link 122b and the fifth connection hole 112a at the first tab 108a of the first operation lever 102a. In addition, a pin-shaped first coupling member (for example, bolt) (not illustrated) passes through the first slit 42 of the first half body 32a, the third connection hole 308, the second coupling holes 128b, the sixth connection holes 114a, and the first slit 42 of the second half body 32b. A retaining member (for example, a nut) is attached to the first coupling member. It is to be noted that the fourth end 124d and the second tabs 110a are relatively rotatable with respect to the block 304.

The second toggle mechanism 120b is coupled to the casing 20, the second operation lever 102b, and the shaft-side slider 302. Specifically, the second toggle mechanism 120b has a pair of left and right third links 122c and a pair of left and right fourth links 122d. The third link 122c on the left side and the fourth link 122d on the left side are rotatably connected to each other. The third link 122c on the right side and the fourth link 122d on the right side are rotatably connected to each other. Each of the pair of third links 122c has a fifth end 124e and a sixth end 124f, a pin-shaped third engagement protrusion 126c is provided at the fifth end 124e, and a pin-shaped fourth engagement protrusion 126d is provided at the sixth end 124f. Each of the pair of fourth links 122d has a seventh end 124g and an eighth end 124 h, and a third coupling hole 128 c is formed in the seventh end 124g. The third coupling hole 128c overlaps the seventh connection hole 112b in the width direction. A fourth coupling hole 128d is formed in the eighth end 124h. The eighth end 124h of the fourth link 122d is interposed between the fourth tab 110b of the second operation lever 102b and the block 304 of the shaft-side slider 302 in the width direction. Therefore, the eighth connection holes 114b at the fourth tabs 110b, the fourth coupling holes 128d at the eighth ends 124h, and the fourth connection hole 310 at the block 304 overlap each other in the width direction.

The third engagement protrusion 126c at the fifth end 124e of the third link 122c is rotatably engaged with the second connection hole 48 formed in the casing 20. The fourth engagement protrusion 126d at the sixth end 124f of the third link 122c is rotatably engaged with the third coupling hole 128 c at the seventh end 124g of the fourth link 122d and the seventh connection hole 112b at the third tab 108b of the second operation lever 102b. In addition, a pin-shaped second coupling member (for example, a bolt) (not illustrated) passes through the second slit 44 of the first half body 32a, the fourth connection hole 310, the fourth coupling hole 128d, the eighth connection hole 114b, and the second slit 44 of the second half body 32b. A retaining member (for example, a nut) is attached to the second coupling member. It is to be noted that the eighth end 124h and the fourth tab 110b are relatively rotatable with respect to the block 304.

The proximal end of the driven shaft 320 is inserted into the distal end of the sleeve 306 of the shaft-side slider 302. Therefore, when the shaft-side slider 302 moves toward the distal end or the proximal end along the axial direction, the driven shaft 320 moves integrally with the shaft-side slider 302.

In the illustrated example, the driven shaft 320 is accommodated in the hollow interior of the shaft member 230. As the user operates the first operation lever 102a and the second operation lever 102b, power is transmitted to the driven shaft 320 via the shaft-side slider 302. As a result, the driven shaft 320 moves inside the shaft member 230. As described above, in the present embodiment, the driven shaft 320 is movably accommodated in the shaft member 230.

A cylindrical spacer 130 is attached to the proximal end of the shaft member 230. The outer diameter of the cylindrical spacer 130 is slightly larger than the outer diameter of the shaft member 230. The cylindrical spacer 130 is fitted into the second fixing hole 58, and the shaft member 230 is fitted into the first fixing hole 30. Since the outer diameter of the cylindrical spacer 130 is larger than the inner diameters of the first fixing hole 30 and the second guide hole 56, the cylindrical spacer 130 is prevented from falling out of the second fixing hole 58 and the second guide hole 56 via the first fixing hole 30. Therefore, the shaft member 230 is also prevented from falling out of the first fixing hole 30. As understood from the above, the cylindrical spacer 130 prevents the shaft member 230 from falling out of the first fixing hole 30.

The medical device 10 includes a cutter 150 (see FIG. 1). As illustrated in FIGS. 2 and 3, the operation unit 100 includes a cutter moving mechanism 152 for moving the cutter 150 along the axial direction of the shaft member 230. The cutter moving mechanism 152 includes a cutter moving operation member 154 illustrated in FIGS. 2 to 4.

In the illustrated example, the cutter moving operation member 154 is a lever 155 provided on the first side surface 22a of the casing 20. However, the cutter moving operation member 154 is not limited to the lever 155. For example, the cutter moving operation member 154 may be a slide switch. The cutter moving operation member 154 may be disposed on the second side surface 22b of the casing 20.

The lever 155 has a first triangular piece 156a and a second triangular piece 156b each having a substantially triangular shape when viewed from the width direction. Each of the first triangular piece 156a and the second triangular piece 156b has a support end 158, a free end 160, and a constrained end 162. A support hole 153 overlapping the support hole 50 of the casing 20 is formed in the support end 158. The support end 158 is rotatably connected to the support holes 50 and 153 via a support member (for example, bolt and nut) (not illustrated). The free end 160 is exposed from the first side surface 22a of the casing 20. A curved wall portion 164 extending in the width direction while being curved is provided between the support end 158 and the free end 160. The curved wall portion 164 of the first triangular piece 156a and the curved wall portion 164 of the second triangular piece 156b are connected to form a push portion 166 having a semi-cylindrical shape.

A pair of arc-shaped slits 168 cut out in an arc shape is formed in the vicinity of the constrained end 162. The constrained end 162 passes through the gap 38 and is exposed to the second side surface 22b of the casing 20, but is covered with a changeover switch 200 provided on the second side surface 22b. Therefore, it is not visually recognized from the outside of the casing 20.

The cutter moving mechanism 152 further includes a cutter-side slider 170 and a wire 180. The cutter-side slider 170 includes a first column 172 extending along the axial direction and a second column 174 extending along the thickness direction. A pair of push-out pins 176 is provided at the distal end of the first column 172. The pair of push-out pins 176 extends along the width direction, passes through the pair of arc-shaped slits 168, respectively, and is inserted into the third slide hole 64 of the first half body 32a and the third slide hole 64 of the second half body 32b, respectively. In other words, the constrained end 162 of the lever 155 (cutter moving operation member 154) restrains the pair of push-out pins 176 respectively passed through the pair of arc-shaped slits 168.

The second column 174 is inserted into the sleeve 306 of the shaft-side slider 302. The lower end of the second column 174 is exposed from the first opening 312 and inserted into the first slide hole 60. The upper end of the second column 174 is exposed from the second opening 314 and is inserted into the second slide hole 62 together with the first column 172. The second column 174 is not restrained by the sleeve 306. Due to such non-restraint, the other of the shaft-side slider 302 and the cutter-side slider 170 is prevented from moving following the movement of one of the shaft-side slider 302 and the cutter-side slider 170 in the casing 20.

The second column 174 holds the proximal end of the wire 180. The distal end of the wire 180 passes through the sleeve 306 and the driven shaft 320 and is connected to the proximal end of the cutter 150. Therefore, when the cutter-side slider 170 moves inside the casing 20, the wire 180 and the cutter 150 integrally move in the same direction as the cutter-side slider 170.

A second return spring (not illustrated) is accommodated in the casing 20. The second return spring biases the cutter-side slider 170 toward the proximal end.

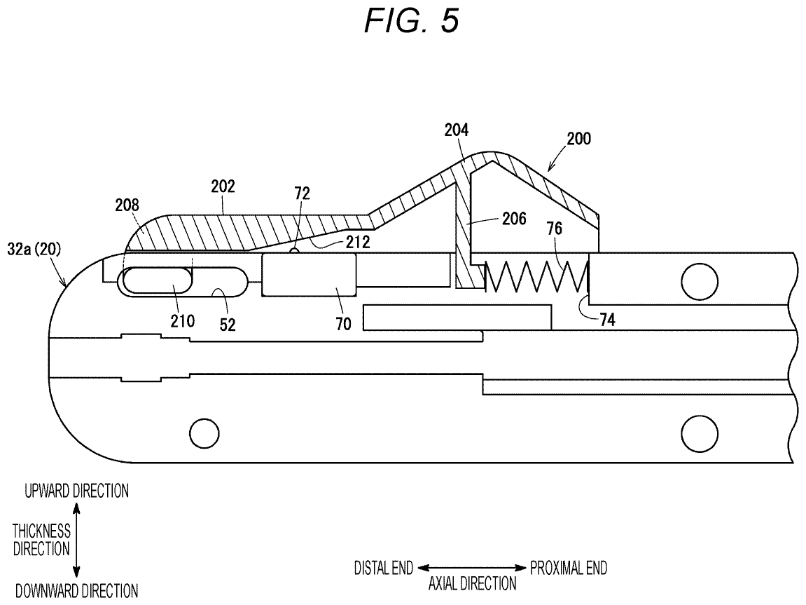

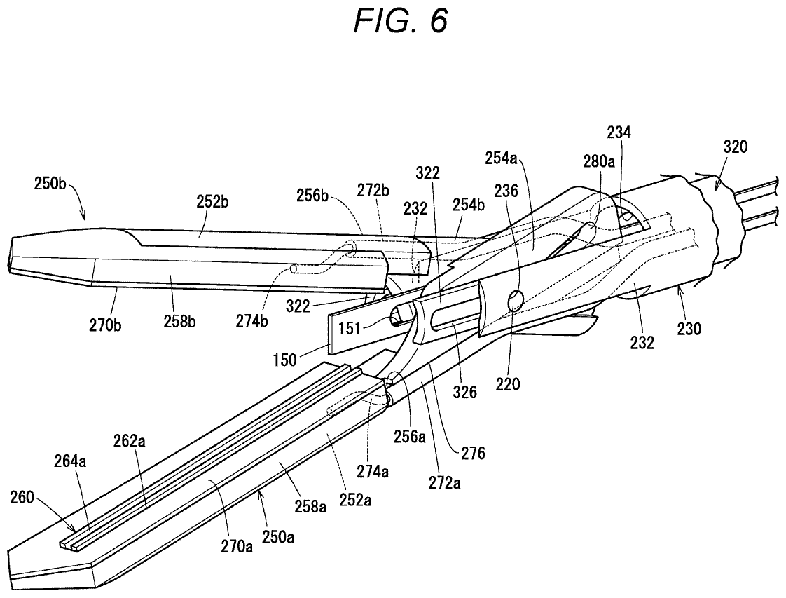

As illustrated in FIG. 6, the first jaw 250a has a first electrode 270a, and the second jaw 250b has a second electrode 270b. As illustrated in FIG. 5, the operation unit 100 includes a changeover switch 200 that switches energization or deenergization between the first electrode 270a and the second electrode 270b. The changeover switch 200 is provided on the second side surface 22b of the casing 20. That is, in the medical device 10 in the illustrated example, the lever 155 (cutter moving operation member 154) and the changeover switch 200 are line-symmetric with the axis of the casing 20 as the symmetry center. When the lever 155 (cutter moving operation member 154) is disposed on the second side surface 22b, the changeover switch 200 may be disposed on the first side surface 22a. The changeover switch 200 may be a lever.

In the illustrated example, the changeover switch 200 is a slide switch movable along the axial direction. The changeover switch 200 includes a nose portion 202, a roof portion 204, and a leg portion 206. The nose portion 202 protrudes from the distal end of the roof portion 204 and extends along the axial direction. The distal end of the nose portion 202 has a pair of cover portions 208 protruding downward and covering a part of the outer surface of the casing 20 in the width direction. Each of the pair of cover portions 208 has an engagement protrusion 210 on an inner surface facing the casing 20. The engagement protrusion 210 is slidably engaged with the engagement hole 52, whereby the changeover switch 200 is prevented from falling off from the casing 20. The nose portion 202 further includes an inclined surface 212 formed on an inner surface facing the casing 20. The inclined surface 212 is a surface inclined upward from the distal end toward the proximal end. When the nose portion 202 is located at the most distal end, the inclined surface 212 is separated from the button 72 of the switch box 70.

The leg portion 206 extends downward from the ceiling surface inside the roof portion 204. The lower end of the leg portion 206 is inserted into the first accommodation hole 74. The first return spring 76 accommodated in the first accommodation hole 74 biases the changeover switch 200 toward the distal end via the leg portion 206. The interior of the roof portion 204 is hollow to such an extent that the constrained end 162 of the cutter moving operation member 154 can be accommodated.

As illustrated in FIGS. 2 and 3, the proximal-side dome portion 24 further includes a second accommodation hole 82 that is in communication with the internal space 40 and the insertion hole 28. As illustrated in FIG. 2, the operation unit 100 includes a stopper 86 accommodated in the second accommodation hole 82. In the illustrated example, the stopper 86 also serves as the coil spring 88. The coil spring 88 biases the power transmission member 300 (the shaft-side slider 302 or the driven shaft 320) toward the distal end. Alternatively, the stopper 86 may be a collar member. In this case, the coil spring 88 is preferably accommodated in the casing 20 separately from the stopper 86. The coil spring 88 has a through hole 90 extending in the axial direction.

As illustrated in FIG. 6, the shaft member 230 has a pair of first extension portions 232. The pair of first extension portions 232 extends from the distal end of the shaft member 230 along the axial direction of the shaft member 230. A pair of first cutout grooves 234 is formed between the pair of first extension portions 232. The pair of first cutout grooves 234 is disposed at positions separated from each other by 180° in the circumferential direction. The pair of first cutout grooves 234 extends along the axial direction of the shaft member 230.

A pair of support holes 236 is formed in the pair of first extension portions 232, respectively. The support hole 236 extends along the width direction of the casing 20 and penetrates the first extension portion 232. Center positions of the pair of support holes 236 are shifted by an angle of 90° in the circumferential direction of the shaft member 230 with respect to centers of the pair of first cutout grooves 234. Both end portions of the rotation shaft 220 described later in the axial direction are fitted into the pair of support holes 236, respectively. Due to the fitting, the rotation shaft 220 is positioned and fixed with respect to the shaft member 230.

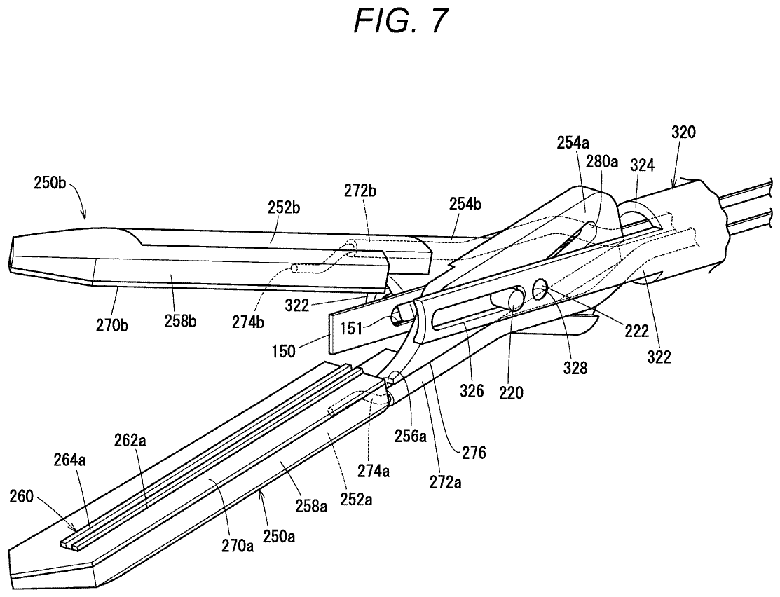

As illustrated in FIG. 7 in which the shaft member 230 is not illustrated, the driven shaft 320 has a pair of second extension portions 322. The pair of second extension portions 322 extends from the distal end of the driven shaft 320 along the axial direction of the driven shaft 320. A pair of second cutout grooves 324 is formed between the pair of second extension portions 322. The pair of second cutout grooves 324 is disposed at positions separated from each other by 180° in the circumferential direction. The pair of second cutout grooves 324 extends along the axial direction of the driven shaft 320. The pair of second extension portions 322 is covered by the pair of first extension portions 232. The pair of second cutout grooves 324 overlaps the pair of first cutout grooves 234.

A pair of guide grooves 326 and a pair of pin attachment holes 328 are formed on the pair of second extension portions 322, respectively. The pair of guide grooves 326 extends along the axial direction of the driven shaft 320 and penetrates from the outer surfaces to the inner surfaces of the pair of second extension portions 322. The pair of pin attachment holes 328 is located proximal to the pair of guide grooves 326. Each of the pair of pin attachment holes 328 is a circular through hole penetrating from the outer surface to the inner surface of the corresponding one of the pair of second extension portions 322. The center positions of the pair of guide grooves 326 and the pair of pin attachment holes 328 are shifted by 90° in the circumferential direction of the driven shaft 320 with respect to the center of the pair of second cutout grooves 324.

The rotation shaft 220 having a cylindrical shape is inserted into each of the pair of guide grooves 326. The rotation shaft 220 moves in the guide grooves 326 relative to the driven shaft 320 along with the axial displacement of the first jaw 250a and the second jaw 250b. Note that, as described above, the ends of the rotation shaft 220 are fitted into the pair of support holes 236 formed in the pair of first extension portions 232 of the shaft member 230.

The ends of an opening and closing pin 222 are inserted into the pair of pin attachment holes 328. The opening and closing pin 222 is a pin fixed to the distal end of the driven shaft 320. When the first jaw 250a and the second jaw 250b are displaced along the axial direction, the opening and closing pin 222 is displaced relative to the first jaw 250a and the second jaw 250b.

As illustrated in FIGS. 6 to 8, the first jaw 250a integrally includes a first support portion 252a and a first proximal end portion 254a connected to the proximal side of the first support portion 252a. The first support portion 252a has a first notch 256a.

The first jaw 250a supports a first main body 258a made of a plate-like insulator and a first electrode 270a. The first main body 258a and the first electrode 270a are supported by the first support portion 252a in a stacked state. The first main body 258a is interposed between the first support portion 252a and the first electrode 270a, and electrically insulates the first support portion 252a from the first electrode 270a.

The first main body 258a has a spacer 260 protruding toward the second support portion 252b. A part of the spacer 260 is exposed from a first electrode-side slit 262a formed in the first electrode 270a and protrudes toward the second support portion 252b. The spacer 260 is formed with a first cutter groove 264a. The first cutter groove 264a extends in the axial direction of the first jaw 250a and guides the movement of the cutter 150 in the axial direction. The spacer 260 is interposed between the cutter 150 inserted into the first cutter groove 264a and the first electrode 270a, and electrically insulates the cutter 150 from the first electrode 270a.

As illustrated in FIGS. 6 and 7, a first power line 272a is electrically connected to the first electrode 270a. Specifically, the distal end of the first power line 272a passes through the first notch 256a formed in the first support portion 252a, and then is inserted into a first piercing hole 274a formed in the first main body 258a. In the first piercing hole 274a, a part of a surface of the first electrode 270a facing the first main body 258a is exposed. The first power line 272a is joined to a portion of the first electrode 270a exposed in the first piercing hole 274a.

At the distal end of the first power line 272a, a portion exposed from the first notch 256a is disposed along the first proximal end portion 254a. In the illustrated example, a recessed groove 276 is formed in the first proximal end portion 254a, and the distal end of the first power line 272a is inserted into the recessed groove 276. The proximal side of the first power line 272a passes through, for example, the hollow interior of the driven shaft 320 and is exposed from the hollow interior of the driven shaft 320 inside the casing 20. The proximal side of the first power line 272a further passes through the through hole 90 of the coil spring 88 (stopper 86) and the insertion hole 28 in the proximal-side dome portion 24 of the casing 20, is exposed to the outside of the casing 20, and is connected to a predetermined power supply device.

A first bearing hole 278a and a first slide groove 280a are formed in the first proximal end portion 254a. The rotation shaft 220 is rotatably passed through the first bearing hole 278a.

The position and the shape of the second jaw 250b are rotationally symmetric with the position and the shape of the first jaw 250a with the central axis C of the shaft member 230 as a rotation center. Therefore, for components of the second jaw 250b corresponding to the components of the first jaw 250a, names obtained by replacing “first” with “second” and reference signs obtained by replacing “a” with “b” are used, and the detailed description thereof will be omitted.

A second cutter groove 264b and a second electrode-side slit 262b are formed in a second main body 258b and a second electrode 270b, respectively. The second electrode-side slit 262b overlaps the second cutter groove 264b. The second electrode-side slit 262b is wider than the first cutter groove 264a. Thus, when the cutter 150 moves into the first cutter groove 264a, the side surface of the cutter 150 is prevented from coming into contact with the inner surface of the second electrode-side slit 262b. In other words, the side surface of the cutter 150 and the side surface of the second electrode-side slit 262b are not in contact with each other. Due to such non-contact, the cutter 150 and the second electrode 270b are electrically insulated.

As illustrated in FIGS. 6 and 7, the distal end of a second power line 272b passes through a second notch 256b formed in the second support portion 252b, is then inserted into a second piercing hole 274b formed in the second main body 258b, and is electrically connected to the second electrode 270b. The proximal side of the second power line 272b passes through, for example, the hollow interior of the driven shaft 320 and is exposed from the hollow interior of the driven shaft 320 inside the casing 20. The proximal side of the second power line 272b further passes through the through hole 90 of the coil spring 88 (stopper 86) and the insertion hole 28 in the proximal-side dome portion 24 of the casing 20, is exposed to the outside of the casing 20, and is connected to a predetermined power supply device.

As illustrated in FIG. 7, when the first proximal end portion 254a of the first jaw 250a and the second proximal end portion 254b of the second jaw 250b are inserted into the second cutout groove 324 of the driven shaft 320 and held between the pair of second extension portions 322, the first bearing hole 278a and the second bearing hole 278b overlap the pair of guide grooves 326. The rotation shaft 220 passes through one of the pair of support holes 236, one of the pair of guide grooves 326, the first bearing hole 278a, an elongated hole 151 (described later) formed in the cutter 150, the second bearing hole 278b, the other of the pair of guide grooves 326, and the other of the pair of support holes 236. As described above, the first proximal end portion 254a of the first jaw 250a and the second proximal end portion 254b of the second jaw 250b are connected with the rotation shaft 220.

When the first proximal end portion 254a of the first jaw 250a and the second proximal end portion 254b of the second jaw 250b are held between the pair of second extension portions 322 of the driven shaft 320, the pair of pin attachment holes 328 overlaps at predetermined positions of the first slide groove 280a and the second slide groove 280b. Therefore, the opening and closing pin 222 passes through one of the pair of pin attachment holes 328, the first slide groove 280a, the elongated hole 151, the second slide groove 280b, and the other of the pair of pin attachment holes 328. As described later, as the opening and closing pin 222 moves relative to the first slide groove 280a and the second slide groove 280b, the first jaw 250a and the second jaw 250b are opened and closed.

The cutter 150 is disposed between the first proximal end portion 254a and the second proximal end portion 254b. The cutter 150 extends along the axial direction of the shaft member 230 and the driven shaft 320. The cutter 150 is positioned on the proximal side in an initial state. Here, the operation unit 100 is provided with the cutter moving operation member 154 as described above. When the user operates the cutter moving operation member 154, the cutter 150 advances toward the distal end in the axial direction or retracts toward the proximal end in the axial direction.

As illustrated in FIGS. 6 to 8, the cutter 150 has the elongated hole 151 penetrating along the thickness direction of the cutter 150. The rotation shaft 220 and the opening and closing pin 222 are inserted into the elongated hole 151.

The medical device 10 according to the present embodiment is basically configured as described above. Next, vessel harvesting using the medical device 10 will be described. Note that the user in the following description is, for example, a medical worker, typically a doctor.

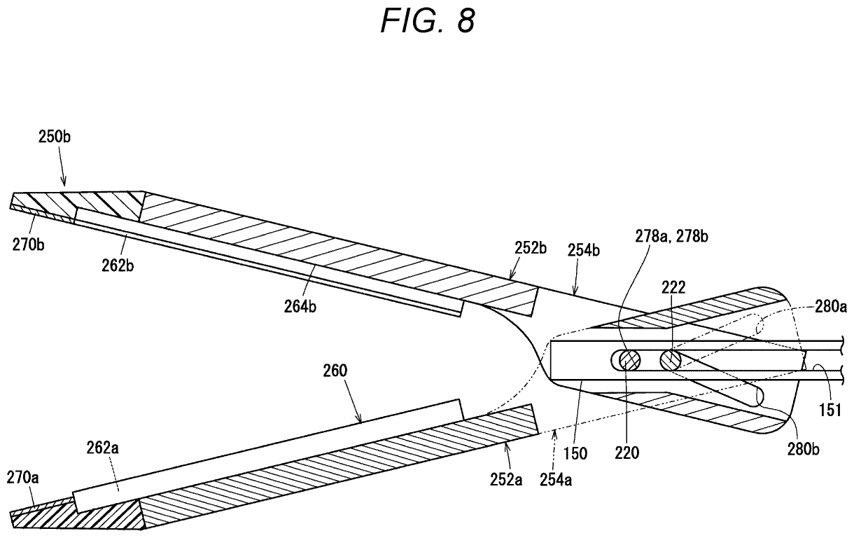

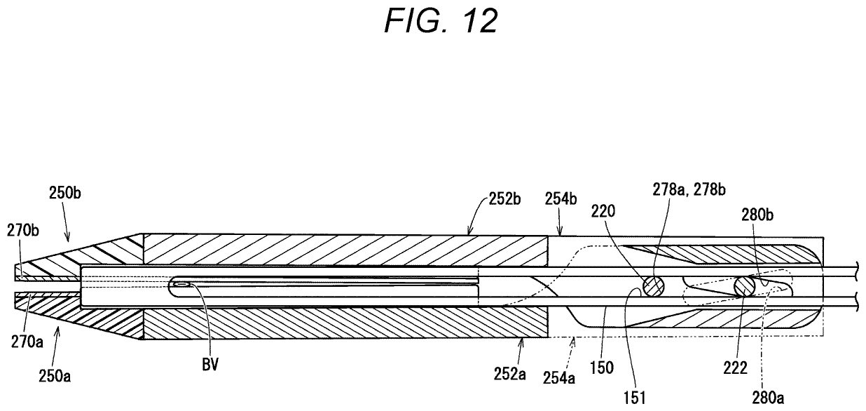

To harvest a blood vessel such as a branch vessel BV (see FIG. 12) from the body of a patient, the medical device 10 and an imaging device are inserted into the body of the patient. The medical device 10 is brought close to the branch vessel BV to be harvested, and the first jaw 250a and the second jaw 250b are brought into an open state illustrated in FIG. 8. Specifically, the user inserts, for example, his/her little finger into the first finger hole 106a (see FIGS. 1 to 3) of the first operation lever 102a, and inserts, for example, his/her thumb into the second finger hole 106b of the second operation lever 102b. Next, the user moves his/her thumb (first finger hook portion 104a) and little finger (second finger hook portion 104b) in directions away from each other. As a result, the first operation lever 102a rotates about the pin-shaped first coupling member (not illustrated) inserted into the third connection hole 308 as a rotation center, and the second operation lever 102b rotates about the pin-shaped second coupling member (not illustrated) inserted into the fourth connection hole 310 as a rotation center. The rotation directions of the first operation lever 102a and the second operation lever 102b are directions in which each of the first finger hook portion 104a and the second finger hook portion 104b is separated from the casing 20.

As the first operation lever 102a and the second operation lever 102b rotate as described above, the power transmission member 300 (the shaft-side slider 302 and the driven shaft 320) integrally moves toward the proximal side. This is because the first operation lever 102a and the shaft-side slider 302 are connected via the first toggle mechanism 120a, and the second operation lever 102b and the shaft-side slider 302 are connected via the second toggle mechanism 120b.

Here, the opening and closing pin 222 (see FIGS. 7 and 8) is immovable with respect to the driven shaft 320. Therefore, the opening and closing pin 222 relatively moves toward the distal side with respect to the first slide groove 280a of the first jaw 250a and the second slide groove 280b of the second jaw 250b. In other words, the first jaw 250a and the second jaw 250b are relatively displaced toward the proximal side of the second cutout groove 324. With this relative movement, the first jaw 250a and the second jaw 250b rotate about the rotation shaft 220 as a rotation center. That is, the first support portion 252a of the first jaw 250a and the second support portion 252b of the second jaw 250b are separated from each other. Therefore, the first jaw 250a and the second jaw 250b are in the open state illustrated in FIG. 8.

As described above, as the user rotates the first operation lever 102a and the second operation lever 102b in the direction in which the first finger hook portion 104a and the second finger hook portion 104b are opened, the first jaw 250a and the second jaw 250b are relatively moved to the proximal side and are changed to the open state. At this time, the coil spring 88 is pressed and compressed by the block 304 of the shaft-side slider 302. The coil spring 88 stops the movement of the block 304 in the casing 20. With the stop of the movement, the rotation of the first operation lever 102a and the second operation lever 102b is stopped, and the rotation of the first jaw 250a and the second jaw 250b is stopped.

At this point, the cutter 150 is most retracted and positioned on the proximal side. The user places the first jaw 250a and the second jaw 250b in the open state in the vicinity of the predetermined branch vessel BV while observing with the imaging device.

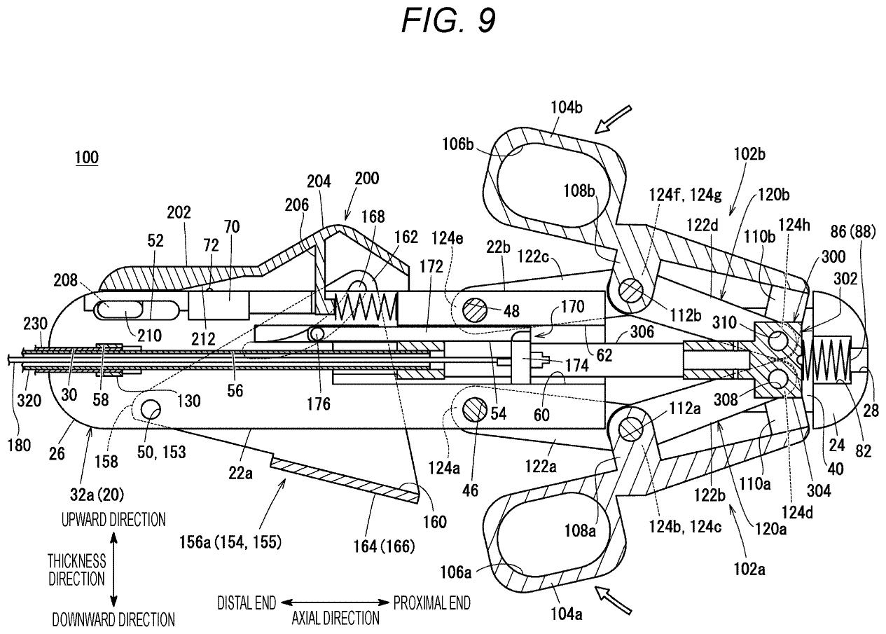

Next, as illustrated in FIG. 9, the user brings the first support portion 252a of the first jaw 250a and the second support portion 252b of the second jaw 250b close to each other, and changes the first jaw 250a and the second jaw 250b to the closed state. Specifically, the user moves his/her thumb (first finger hook portion 104a) and little finger (second finger hook portion 104b) in a direction of approaching each other. As a result, the first operation lever 102a rotates about the pin-shaped first coupling member (not illustrated) inserted into the third connection hole 308 as a rotation center, and the second operation lever 102b rotates about the pin-shaped second coupling member (not illustrated) inserted into the fourth connection hole 310 as a rotation center. The rotation directions of the first operation lever 102a and the second operation lever 102b are directions in which each of the first finger hook portion 104a and the second finger hook portion 104b approaches the casing 20.

As the first operation lever 102a and the second operation lever 102b rotate as described above, the power transmission member 300 (the shaft-side slider 302 and the driven shaft 320) integrally moves toward the distal side. Therefore, the opening and closing pin 222 moves to the proximal side relative to the first slide groove 280a of the first jaw 250a and the second slide groove 280b of the second jaw 250b. In other words, the first jaw 250a and the second jaw 250b are relatively displaced toward the distal side of the second cutout groove 324. With this relative movement, the first jaw 250a and the second jaw 250b rotate about the rotation shaft 220 as a rotation center. That is, the first support portion 252a of the first jaw 250a and the second support portion 252b of the second jaw 250b are close to each other. Therefore, the first jaw 250a and the second jaw 250b are in the closed state illustrated in FIG. 12.

As described above, as the user rotates the first operation lever 102a and the second operation lever 102b in the closing direction, the first jaw 250a and the second jaw 250b move toward the distal side relative to the shaft member 230 and are changed to the closed state. As a result, the branch vessel BV (see FIG. 12) is pinched between the first support portion 252a and the second support portion 252b. FIG. 12 illustrates a state in which the cutter 150 has moved to the distal end. However, at the time point at which the branch vessel BV is pinched between the first support portion 252a and the second support portion 252b, the cutter 150 has not moved from the position illustrated in FIG. 8 and is located at the proximal end.

At this point, the spacer 260 exposed from the first electrode-side slit 262a contacts the second electrode 270b. With this contact, the first electrode 270a and the second electrode 270b are slightly separated away from each other. In addition, the first cutter groove 264a and the second cutter groove 264b face each other.

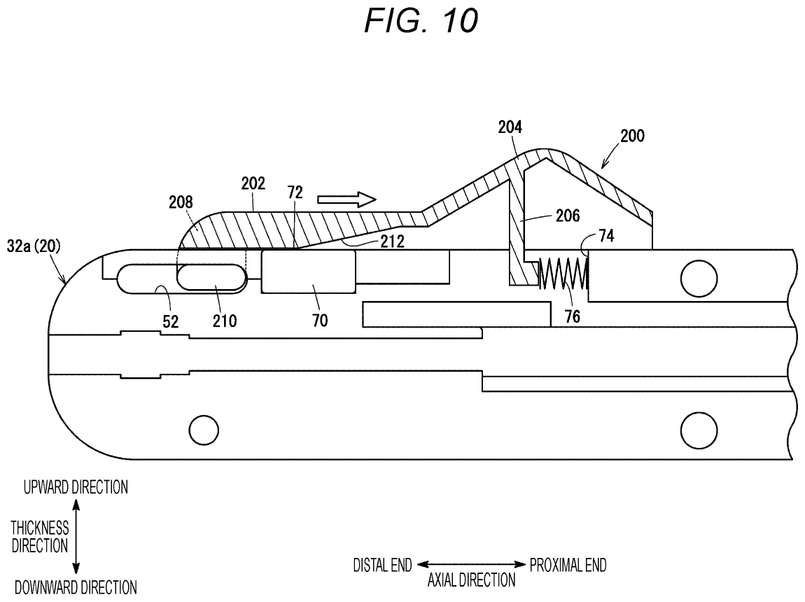

Next, while maintaining the first jaw 250a and the second jaw 250b in the closed state, the user operates the changeover switch 200 provided at the operation unit 100 as illustrated in FIG. 10, for example. The changeover switch 200 is biased toward the distal side by the first return spring 76 via the leg portion 206. In this case, the inclined surface 212 of the changeover switch 200 is not in contact with the button 72 of the switch box 70. The user places, for example, his/her index finger on the roof portion 204 of the changeover switch 200 in this state and pulls the roof portion toward the proximal side. As a result, the changeover switch 200 moves toward the proximal side while compressing the first return spring 76 with the leg portion 206. The engagement protrusion 210 of the nose portion 202 is engaged with the engagement hole 52 as described above, and thus, the changeover switch 200 is guided by the engagement hole 52 and smoothly moves to the proximal side.

As the changeover switch 200 moves to the proximal side, the inclined surface 212 comes into contact with the button 72. Thus, a high-frequency current is supplied to the first electrode 270a and the second electrode 270b via the first power line 272a and the second power line 272b (see FIGS. 6 and 7). As a result, the branch vessel BV (see FIG. 12) pinched between the first support portion 252a and the second support portion 252b is cauterized and hemostasis is performed.

Thereafter, the user releases his/her index finger from the roof portion 204 (see FIG. 10). Accordingly, the first return spring 76 compressed by the leg portion 206 expands, and biases the changeover switch 200 toward the distal side via the leg portion 206. As a result, the changeover switch 200 moves to the distal side and returns to the original position (see FIG. 5). Also at this time, the engagement protrusion 210 of the nose portion 202 is guided to the engagement hole 52.

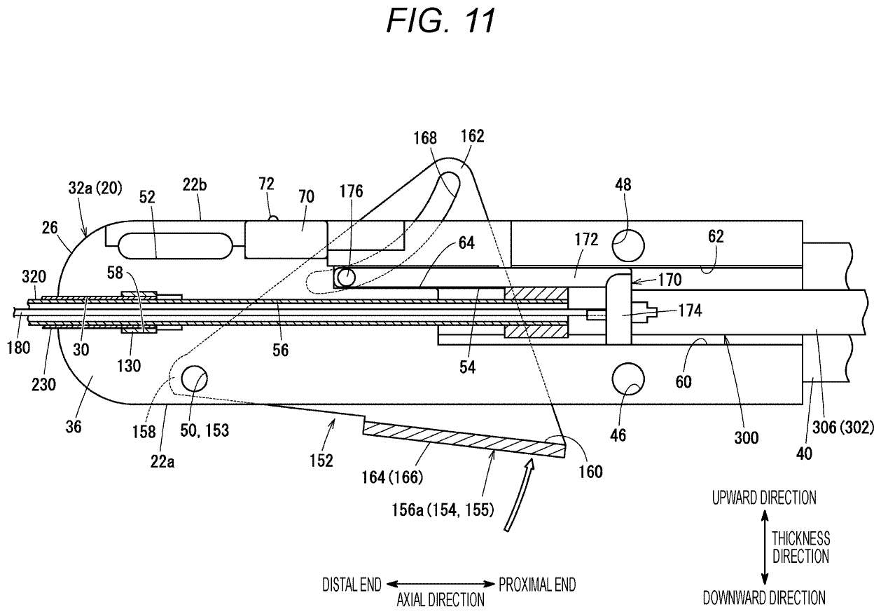

Next, the user operates the cutter moving operation member 154 as illustrated in FIG. 11 while maintaining the first jaw 250a and the second jaw 250b in the closed state. Specifically, the user pushes up the push portion 166 of the cutter moving operation member 154 toward the second side surface 22b with his/her ring finger. As a result, the cutter moving operation member 154 rotates about the bolt or the like inserted into the support hole 50. The rotation direction of the cutter moving operation member 154 is a direction in which the free end 160 approaches the first side surface 22a of the casing 20.

At the constrained end 162 of the cutter moving operation member 154, the pair of push-out pins 176 of the cutter-side slider 170 passes through the pair of arc-shaped slits 168, respectively. Therefore, when the cutter moving operation member 154 rotates as described above, the pair of push-out pins 176 is pushed toward the distal end by the inner surfaces of the pair of arc-shaped slits 168. As a result, the cutter-side slider 170 moves toward the distal end while sliding in the first slide hole 60, the second slide hole 62, and the third slide hole 64. As described above, the second column 174 of the cutter-side slider 170 is not restrained by the sleeve 306 of the shaft-side slider 302, and thus, the shaft-side slider 302 is prevented from moving following the movement of the cutter-side slider 170 in the casing 20.

Therefore, the cutter 150 is pushed out by the wire 180 connected to the cutter 150 and the cutter-side slider 170, and inserted into the first cutter groove 264a and the second cutter groove 264b while moving from the position on the proximal side illustrated in FIG. 8 to the position on the distal side illustrated in FIG. 12. The cutter 150 advances along the first cutter groove 264a and the second cutter groove 264b, and cuts the branch vessel BV pinched between the first support portion 252a and the second support portion 252b. If necessary, the medical device 10 may be further advanced in the body of the patient to cut another branch vessel or exfoliate surrounding tissues.

After the cutting of the branch vessel in the desired region is completed, the medical device 10 and the imaging device are withdrawn from the body of the patient. Thus, the main operation of the vessel harvesting is completed.

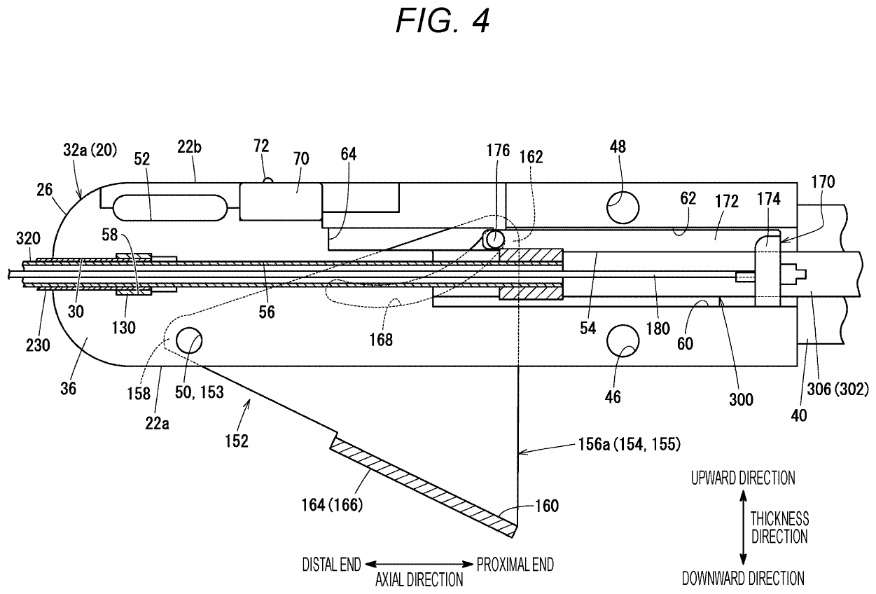

When the user releases his/her finger from the push portion 166, the cutter-side slider 170 is biased in the proximal direction by the second return spring in the casing 20. As a result, the cutter-side slider 170 moves to the proximal end, by which the cutter-side slider 170 and the cutter moving operation member 154 (lever 155) return to the original positions illustrated in FIG. 4.

The present embodiment provides the following effects.

As illustrated in FIG. 1, the medical device 10 includes an operation unit 100 operated by a user, a shaft member 230 extending in the distal direction from the operation unit 100, and a first jaw 250a and a second jaw 250b provided to be openable and closable at the distal end of the shaft member 230. The operation unit 100 includes a casing 20, a first operation lever 102a, and a second operation lever 102b. The first operation lever 102a has an annular first finger hook portion 104a in which a first finger hole 106a is formed. The second operation lever 102b has an annular second finger hook portion 104b in which a second finger hole 106b is formed. The first finger hole 106a is a hole for the user to insert his/her finger, and the second finger hole 106b is a hole for the user to insert his/her finger different from the finger to be inserted into the first finger hole 106a. The first operation lever 102a and the second operation lever 102b are rotatable with respect to the casing 20 and face each other across the casing 20.

The medical device 10 includes a power transmission member 300. The power transmission member 300 is movable along the axial direction of the shaft member 230, and rotates the first jaw 250a or the second jaw 250b as it moves along the axial direction with respect to the shaft member 230. Due to this rotation, the first jaw 250a and the second jaw 250b are opened and closed.

That is, the power transmission member 300 moves in the axial direction with respect to the shaft member 230 in conjunction with the rotation operation of the first operation lever 102a and the second operation lever 102b with respect to the casing 20. Based on this movement, the first jaw 250a and the second jaw 250b are opened and closed.

In this manner, the user inserts his/her fingers into the first operation lever 102a and the second operation lever 102b, respectively, while holding (gripping) the casing 20 in the palm, and with this state, moves the fingers to apply rotational force to the first operation lever 102a and the second operation lever 102b, by which the first jaw 250a and the second jaw 250b can be opened and closed. The user can easily put strength in his/her hand and can easily move the fingers, whereby the operability of the medical device 10 is improved.

As understood with reference to FIGS. 3, 7, 9, and 12, in the above-described aspect, as the first operation lever 102a rotates in such a manner that the first finger hook portion 104a approaches the casing 20, and the second operation lever 102b rotates in such a manner that the second finger hook portion 104b approaches the casing 20, the power transmission member 300 moves toward the distal end along the axial direction of the shaft member 230. Further, the first jaw 250a and the second jaw 250b are brought into a closed state. On the other hand, as the first operation lever 102a rotates in such a manner that the first finger hook portion 104a is separated from the casing 20 and the second operation lever 102b rotates in such a manner that the second finger hook portion 104b is separated from the casing 20, the power transmission member 300 moves toward the proximal end along the axial direction of the shaft member 230. Further, the first jaw 250a and the second jaw 250b are brought into an open state.

As described above, in the above aspect, when both the first operation lever 102a and the second operation lever 102b are brought into the closed state of approaching the casing 20, the first jaw 250a and the second jaw 250b are brought into the closed state. When both the first operation lever 102a and the second operation lever 102b are brought into the open state of being separated from the casing 20, the first jaw 250a and the second jaw 250b are brought into the open state. That is, the open state and the closed state of the first operation lever 102a and the second operation lever 102b match the open state and the closed state of the first jaw 250a and the second jaw 250b. Therefore, the user can easily recognize that the first jaw 250a and the second jaw 250b have been changed from the open state to the closed state by, for example, operating the first operation lever 102a and the second operation lever 102b to shift them from the open state to the closed state.

As illustrated in FIG. 9, the rotation center of the first operation lever 102a (first coupling member (not illustrated) inserted into the third connection hole 308) is provided at the proximal end portion of the casing 20, and the rotation center of the second operation lever 102b (second coupling member (not illustrated) inserted into the fourth connection hole 310) is provided at the proximal end portion of the casing 20. The first operation lever 102a rotates in such a manner that the first finger hook portion 104a is separated from or approaches the casing 20 on the distal side with respect to the rotation center. Similarly, the second operation lever 102b rotates in such a manner that the second finger hook portion 104b is separated from or approaches the casing 20 on the distal side with respect to the rotation center.

In this case, the user can easily rotate the first operation lever 102a and the second operation lever 102b by moving the fingers that are inserted into the first finger hook portion 104a and the second finger hook portion 104b, respectively.

As illustrated in FIGS. 2 and 3, the operation unit 100 includes a first toggle mechanism 120a connected to the casing 20, the first operation lever 102a, and the power transmission member 300, and a second toggle mechanism 120b connected to the casing 20, the second operation lever 102b, and the power transmission member 300. The first toggle mechanism 120a has a first link 122a and a second link 122b rotatably connected to each other. The first link 122a has a first end 124a rotatably connected to the casing 20 and a second end 124b rotatably connected to the second link 122b, and the second link 122b has a third end 124c rotatably connected to the second end 124b and a fourth end 124d rotatably connected to the power transmission member 300. The first operation lever 102a is connected to a connection point between the second end 124b and the third end 124c.

The second toggle mechanism 120b has a third link 122c and a fourth link 122d rotatably connected to each other. The third link 122c has a fifth end 124e rotatably connected to the casing 20 and a sixth end 124f rotatably connected to the fourth link 122d. The fourth link 122d has a seventh end 124g rotatably connected to the sixth end 124f and an eighth end 124h rotatably connected to the power transmission member 300. The second operation lever 102b is connected to a connection point between the sixth end 124 f and the seventh end 124g.

In other words, the power transmission member 300 constitutes the first toggle mechanism 120a with the first link 122a and the second link 122b, and constitutes the second toggle mechanism 120b with the third link 122c and the fourth link 122d. Therefore, the user can easily move the power transmission member 300 by rotating the first operation lever 102a and the second operation lever 102b with a small force.

As illustrated in FIGS. 1, 6, 7, 8, and 12, the medical device 10 includes a cutter 150 that excises biological tissue such as a branch vessel BV. The operation unit 100 includes a cutter moving operation member 154 for moving the cutter 150 along the axial direction of the shaft member 230 in the casing 20. The cutter moving operation member 154 is located distal to the first finger hook portion 104a or the second finger hook portion 104b.

The casing 20 has a first side surface 22a and a second side surface 22b located opposite to each other with respect to the axis of the casing 20. The first operation lever 102a is disposed on the first side surface 22a. The second operation lever 102b is disposed on the second side surface 22b. The cutter moving operation member 154 is disposed on the first side surface 22a (or the second side surface 22b).

Since the biological tissue such as the branch vessel BV can be excised by the cutter 150, it is easy to harvest the biological tissue. In addition, the user can easily touch the cutter moving operation member 154 with a finger not inserted into the first finger hole 106a and not inserted into the second finger hole 106b while gripping the casing 20 with the palm. Therefore, the user can easily move the cutter 150.

In one aspect, the cutter moving operation member 154 is the lever 155 illustrated in FIGS. 4 and 11. One end of the lever 155 is connected to the casing 20 via a support member on the distal side with respect to the first finger hook portion 104a or the second finger hook portion 104b. The lever 155 is rotatable with the support member as a rotation center in such a manner that the free end 160, which is the other end of the lever 155, approaches or is separated from the casing 20. When the lever 155 rotates in such a manner that the free end 160 of the lever 155 approaches the casing 20, the cutter 150 moves toward the distal end along the axial direction of the shaft member 230.

As described above, the user can easily touch the lever 155 with a finger that is not inserted into the first finger hole 106a and is not inserted into the second finger hole 106b. Moreover, one end (support end 158) of the lever 155 facing the distal end of the casing 20 is connected to the casing 20, and the other end (free end 160) facing the proximal end of the casing 20 is located at the distal end of the casing 20 with respect to the first finger hook portion 104a or the second finger hook portion 104b. Therefore, the lever 155 can be easily rotated by pushing the other end of the lever 155 toward the casing 20 with a finger. Therefore, the user can easily move the cutter 150.

In the above aspect, the first jaw 250a and the second jaw 250b have a first electrode 270a and a second electrode 270b, respectively, for cauterizing the biological tissue as illustrated in FIGS. 6 to 8 and 12. The operation unit 100 includes, in the casing 20, a changeover switch 200 that switches energization or deenergization between the first electrode 270a and the second electrode 270b. The changeover switch 200 is located distal to the first finger hook portion 104a or the second finger hook portion 104b.

The casing 20 has a first side surface 22a and a second side surface 22b located opposite to each other with respect to the axis of the casing 20. The first operation lever 102a is disposed on the first side surface 22a. The second operation lever 102b is disposed on the second side surface 22b. The changeover switch 200 is disposed on the second side surface 22b (or the first side surface 22a).

By energizing the first electrode 270a and the second electrode 270b, it is possible to cauterize the biological tissue pinched between the first jaw 250a and the second jaw 250b. Therefore, when the biological tissue is a blood vessel, hemostasis can be promptly performed when the blood vessel is harvested. In addition, the user can easily touch the changeover switch 200 with a finger not inserted into the first finger hole 106a and not inserted into the second finger hole 106b while gripping the casing 20 with the palm. Therefore, the user can easily switch the energization or deenergization between the first electrode 270a and the second electrode 270b.

As understood with reference to FIGS. 5 and 10, the changeover switch 200 is movable toward the distal end or the proximal end along the axial direction of the shaft member 230.

With this configuration, the user can easily switch energization or deenergization between the first electrode 270a and the second electrode 270b by moving the changeover switch 200 toward the distal end or the proximal end along the axial direction of the casing 20 with the finger not inserted into the first finger hole 106a and not inserted into the second finger hole 106b.

When both the cutter moving operation member 154 and the changeover switch 200 are provided in the casing 20, the changeover switch 200 is disposed on one of the first side surface 22a and the second side surface 22b, and the cutter moving operation member 154 is disposed on the other of the first side surface 22a and the second side surface 22b.

With this configuration, the user can individually touch the cutter moving operation member 154 or the changeover switch 200 with the finger not inserted into the first finger hole 106a and not inserted into the second finger hole 106b while holding the casing 20 with the palm. Therefore, the user can easily move the cutter 150, and can easily switch the energization or the deenergization between the first electrode 270a and the second electrode 270b.

The casing 20 has, at the proximal end, an insertion hole 28 through which the first power line 272a electrically connected to the first electrode 270a and the second power line 272b electrically connected to the second electrode 270b pass.

The proximal end of the casing 20 is a portion that is not touched by a user's finger or palm. Therefore, when the user operates the medical device 10, the first power line 272a and the second power line 272b are prevented from interfering with the operation.

The operation unit 100 includes a stopper 86 that stops the movement of the power transmission member 300 toward the proximal end of the casing 20, the stopper 86 being located proximal to the proximal end of the power transmission member 300 inside the casing 20.

When the power transmission member 300 comes into contact with the stopper 86, the opening operation (or the closing operation) of the first jaw 250a and the second jaw 250b is stopped. Therefore, for example, the first jaw 250a and the second jaw 250b are prevented from being excessively opened, whereby it is easy to insert the first jaw 250a and the second jaw 250b into the body of the living body.

The operation unit 100 includes a coil spring 88 that biases the power transmission member 300 in the distal direction inside the casing 20.

For example, when the user removes his/her fingers from the first finger hole 106a and the second finger hole 106b after the first operation lever 102a and the second operation lever 102b are rotated from the closed state to the open state by the user's operation, the coil spring 88 pushes the power transmission member 300 based on elasticity. As a result, the first operation lever 102a and the second operation lever 102b are changed from the open state to the closed state. Therefore, the user does not need to perform an operation for returning the first operation lever 102a and the second operation lever 102b from the open state to the closed state.

As illustrated in FIGS. 6 to 9, the first jaw 250a and the second jaw 250b have a first electrode 270a and a second electrode 270b, respectively, for cauterizing biological tissue. As illustrated in FIG. 2, the coil spring 88 also serving as the stopper 86 has a through hole 90 through which the first power line 272a (see FIGS. 6 and 7) electrically connected to the first electrode 270 a and the second power line 272b electrically connected to the second electrode 270b pass. The casing 20 has an insertion hole 28 through which the first power line 272a and the second power line 272b pass on the proximal side with respect to the proximal end of the stopper 86.

The first power line 272a and the second power line 272b can pass through the insertion hole 28 formed on the proximal side with respect to the stopper 86 via the through hole 90 of the stopper 86. Therefore, even though the stopper 86 is provided inside the casing 20, the first power line 272a and the second power line 272b can be exposed from the inside to the outside of the casing 20.

As illustrated in FIGS. 2 and 3, the shaft member 230 is a hollow body. Most of the driven shaft 320 which is a part of the power transmission member 300 is inserted into the shaft member 230. The driven shaft 320 can move relative to the shaft member 230.

By inserting a part of the power transmission member 300 into the shaft member 230, the medical device 10 can be downsized.

Hereinafter, modifications of the present invention will be additionally described.

The mechanism that converts the rotational motion of the first operation lever 102a and the second operation lever 102b into the linear motion of the power transmission member 300 may be a mechanism other than the first toggle mechanism 120a and the second toggle mechanism 120b. Examples of such a mechanism include a gear mechanism and a belt mechanism.

The rotation centers of the first operation lever 102a and the second operation lever 102b may be located distal to each of the first finger hook portion 104a and the second finger hook portion 104b.

The rotation direction (opening/closing direction) of the first operation lever 102a and the second operation lever 102b may be different from the opening/closing direction of the first jaw 250a and the second jaw 250b. For example, the first operation lever 102a and the second operation lever 102b may be opened and closed along the lateral direction (width direction), and the first jaw 250a and the second jaw 250b may be opened and closed along the vertical direction (thickness direction).

The above aspect has described the configuration in which both the first jaw 250a and the second jaw 250b rotate, but only one of the first jaw 250a and the second jaw 250b may rotate. For example, the first jaw 250a may be positioned and fixed, and the second jaw 250b may be rotatable with respect to the first jaw 250a. Thus, it is not necessary that both the first jaw 250a and the second jaw 250b rotate.

Note that the present invention is not limited to the above disclosure and can take various configurations without departing from the gist of the present invention.

What is claimed is:

1. A medical device comprising:
an operation unit operated by a user;
a shaft member extending in a distal direction from the operation unit; and
a first jaw and a second jaw provided to be openable and closable at a distal end of the shaft member;
wherein the operation unit comprises: a casing, a first operation lever rotatable with respect to the casing and having a first finger hook portion that is annular and that is formed with a first finger hole into which a finger of the user is inserted, and a second operation lever rotatable with respect to the casing and having a second finger hook portion that is annular and that is formed with a second finger hole into which a second finger of the user different from the finger to be inserted into the first finger hole is inserted, the first operation lever and the second operation lever facing each other across the casing; and
the medical device also comprises a power transmission member that is movable along an axial direction of the shaft member and that rotates at least one of the first jaw and the second jaw in such a manner that the first jaw and the second jaw are opened and closed with the movement in the axial direction with respect to the shaft member, the power transmission member moving in the axial direction with respect to the shaft member in conjunction with rotation operation of the first operation lever and the second operation lever with respect to the casing.

2. The medical device according to claim 1, wherein, as the first operation lever rotates in such a manner that the first finger hook portion approaches the casing, and the second operation lever rotates in such a manner that the second finger hook portion approaches the casing, the power transmission member moves toward the distal end along the axial direction of the shaft member, and the first jaw and the second jaw are in a closed state, and
as the first operation lever rotates in such a manner that the first finger hook portion is separated from the casing, and the second operation lever rotates in such a manner that the second finger hook portion is separated from the casing, the power transmission member moves toward a proximal end along the axial direction of the shaft member, and the first jaw and the second jaw are in an open state.

3. The medical device according to claim 1, wherein rotation centers of the first operation lever and the second operation lever are provided at a proximal end portion of the casing, and
the first operation lever and the second operation lever rotate in such a manner that the first finger hook portion and the second finger hook portion are separated from or approach the casing on a distal side with respect to the rotation centers.

4. The medical device according to claim 2, wherein the operation unit further comprises:
a first toggle mechanism connected to the casing, the first operation lever, and the power transmission member; and
a second toggle mechanism connected to the casing, the second operation lever, and the power transmission member,
the first toggle mechanism includes a first link and a second link rotatably connected to each other,
the first link includes a first end rotatably connected to the casing and a second end rotatably connected to the second link,
the second link includes a third end rotatably connected to the second end and a fourth end rotatably connected to the power transmission member,
the first operation lever is connected to a connection point between the second end and the third end,
the second toggle mechanism includes a third link and a fourth link rotatably connected to each other,
the third link includes a fifth end rotatably connected to the casing and a sixth end rotatably connected to the fourth link,
the fourth link includes a seventh end rotatably connected to the sixth end and an eighth end rotatably connected to the power transmission member, and
the second operation lever is connected to a connection point between the sixth end and the seventh end.

5. The medical device according to claim 1, further comprising a cutter that excises biological tissue, wherein
the operation unit comprises a cutter moving operation member for moving the cutter along the axial direction of the shaft member in the casing,
the cutter moving operation member is located distal to the first finger hook portion or the second finger hook portion,
the casing has a first side surface and a second side surface located opposite to each other with respect to an axis of the casing,
the first operation lever is disposed on the first side surface,
the second operation lever is disposed on the second side surface, and
the cutter moving operation member is disposed on the first side surface or the second side surface.

6. The medical device according to claim 5, wherein the cutter moving operation member is a lever,
one end of the lever is connected to the casing via a support member on a distal side with respect to the first finger hook portion or the second finger hook portion, the lever is rotatable with the support member as a rotation center in such a manner that a free end which is another end of the lever approaches or is separated from the casing, and
the cutter moves toward the distal end along the axial direction of the shaft member by rotating the lever in such a manner that the free end of the lever approaches the casing.

7. The medical device according to claim 1, wherein the first jaw and the second jaw include a first electrode and a second electrode, respectively, for cauterizing biological tissue,
the operation unit further comprises a changeover switch that switches energization or deenergization between the first electrode and the second electrode in the casing,
the changeover switch is located distal to the first finger hook portion or the second finger hook portion,
the casing has a first side surface and a second side surface located opposite to each other with respect to an axis of the casing,
the first operation lever is disposed on the first side surface,
the second operation lever is disposed on the second side surface, and
the changeover switch is disposed on the first side surface or the second side surface.

8. The medical device according to claim 7, wherein the changeover switch is movable toward the distal end or the proximal end along the axial direction of the shaft member.

9. The medical device according to claim 1, further comprising a cutter that excises biological tissue, wherein
the first jaw and the second jaw include a first electrode and a second electrode, respectively, for cauterizing the biological tissue,
the operation unit further comprises, in the casing, a changeover switch that switches energization or deenergization between the first electrode and the second electrode, and a cutter moving operation member for moving the cutter along the axial direction of the casing,
the changeover switch is located distal to the first finger hook portion or the second finger hook portion,
the cutter moving operation member is located distal to the first finger hook portion or the second finger hook portion,
the casing has a first side surface and a second side surface located opposite to each other with respect to an axis of the casing,
the first operation lever is disposed on the first side surface,
the second operation lever is disposed on the second side surface,
the changeover switch is disposed on one of the first side surface and the second side surface, and
the cutter moving operation member is disposed on the other of the first side surface and the second side surface.

10. The medical device according to claim 7, wherein the casing has, at a proximal end, an insertion hole through which a first power line electrically connected to the first electrode and a second power line electrically connected to the second electrode pass.

11. The medical device according to claim 1, wherein the operation unit further comprises a stopper that stops movement of the power transmission member toward a proximal end of the casing, the stopper being located proximal to a proximal end of the power transmission member inside the casing.

12. The medical device according to claim 1, wherein the operation unit further comprises a coil spring that biases the power transmission member in the distal direction inside the casing.

13. The medical device according to claim 11, wherein the first jaw and the second jaw include a first electrode and a second electrode, respectively, for cauterizing biological tissue,
the stopper has a through hole through which a first power line electrically connected to the first electrode and a second power line electrically connected to the second electrode pass, and
the casing has an insertion hole through which the first power line and the second power line pass, the insertion hole being located proximal to a proximal end of the stopper.

14. The medical device according to claim 1, wherein the shaft member is a hollow body, and a part of the power transmission member is inserted into the shaft member so as to be relatively movable with respect to the shaft member.

15. A medical device comprising:
an operation unit operated by a user;
a shaft member extending in a distal direction from the operation unit; and
a first jaw and a second jaw provided to be openable and closable at a distal end of the shaft member;
wherein the operation unit comprises: a casing, a first operation lever rotatable with respect to the casing and having a first finger hook portion that is annular and that is formed with a first finger hole into which a finger of the user is inserted, and a second operation lever rotatable with respect to the casing and having a second finger hook portion that is annular and that is formed with a second finger hole into which a second finger of the user different from the finger to be inserted into the first finger hole is inserted, the first operation lever and the second operation lever facing each other across the casing;
the medical device also comprising: a power transmission member that is movable along an axial direction of the shaft member and that rotates at least one of the first jaw and the second jaw in such a manner that the first jaw and the second jaw are opened and closed with the movement in the axial direction with respect to the shaft member, the power transmission member moving in the axial direction with respect to the shaft member in conjunction with rotation operation of the first operation lever and the second operation lever with respect to the casing; and
wherein the operation unit further comprises:
a first toggle mechanism connected to the casing, the first operation lever, and the power transmission member; and
a second toggle mechanism connected to the casing, the second operation lever, and the power transmission member,
the first toggle mechanism includes a first link and a second link rotatably connected to each other,
the first link includes a first end rotatably connected to the casing and a second end rotatably connected to the second link,
the second link includes a third end rotatably connected to the second end and a fourth end rotatably connected to the power transmission member,
the first operation lever is connected to a connection point between the second end and the third end,
the second toggle mechanism includes a third link and a fourth link rotatably connected to each other,
the third link includes a fifth end rotatably connected to the casing and a sixth end rotatably connected to the fourth link,
the fourth link includes a seventh end rotatably connected to the sixth end and an eighth end rotatably connected to the power transmission member, and
the second operation lever is connected to a connection point between the sixth end and the seventh end.

16. The medical device according to claim 15, further comprising a cutter that excises biological tissue, wherein:
the first jaw and the second jaw include a first electrode and a second electrode, respectively, for cauterizing the biological tissue,
the operation unit further comprises, in the casing, a changeover switch that switches energization or deenergization between the first electrode and the second electrode, and a cutter moving operation member for moving the cutter along the axial direction of the casing,
the changeover switch is located distal to the first finger hook portion or the second finger hook portion,
the cutter moving operation member is located distal to the first finger hook portion or the second finger hook portion,
the casing has a first side surface and a second side surface located opposite to each other with respect to an axis of the casing,
the first operation lever is disposed on the first side surface,
the second operation lever is disposed on the second side surface,
the changeover switch is disposed on one of the first side surface and the second side surface, and
the cutter moving operation member is disposed on the other of the first side surface and the second side surface.

17. The medical device according to claim 16, wherein the casing has, at a proximal end, an insertion hole through which a first power line electrically connected to the first electrode and a second power line electrically connected to the second electrode pass.

18. The medical device according to claim 17, wherein the shaft member is a hollow body, and a part of the power transmission member is inserted into the shaft member so as to be relatively movable with respect to the shaft member.

19. A medical device comprising:
an operation unit operated by a user;
a shaft member extending in a distal direction from the operation unit; and
a first jaw and a second jaw provided to be openable and closable at a distal end of the shaft member;
wherein the operation unit comprises: a casing, a first operation lever rotatable with respect to the casing and having a first finger hook portion that is annular and that is formed with a first finger hole into which a finger of the user is inserted, and a second operation lever rotatable with respect to the casing and having a second finger hook portion that is annular and that is formed with a second finger hole into which a second finger of the user different from the finger to be inserted into the first finger hole is inserted, the first operation lever and the second operation lever facing each other across the casing;
the medical device also comprising: a power transmission member that is movable along an axial direction of the shaft member and that rotates at least one of the first jaw and the second jaw in such a manner that the first jaw and the second jaw are opened and closed with the movement in the axial direction with respect to the shaft member, the power transmission member moving in the axial direction with respect to the shaft member in conjunction with rotation operation of the first operation lever and the second operation lever with respect to the casing;
wherein the operation unit further comprises a stopper that stops movement of the power transmission member toward a proximal end of the casing; and
wherein the first jaw and the second jaw include a first electrode and a second electrode, respectively, for cauterizing biological tissue,
the stopper has a through hole through which a first power line electrically connected to the first electrode and a second power line electrically connected to the second electrode pass, and
the casing has an insertion hole through which the first power line and the second power line pass, the insertion hole being located proximal to a proximal end of the stopper.

20. The medical device according to claim 19, wherein the shaft member is a hollow body, and a part of the power transmission member is inserted into the shaft member so as to be relatively movable with respect to the shaft member.
